(19) United States
(12) Patent Application Publication
(10) Pub. No.: US 2025/0025115 A1
(43) Pub. Date: Jan. 23, 2025

(54) METHOD AND DEVICE FOR MEASURING FEMORAL ANTERIOR ANGLE AND TIBIAL TORSION ANGLE BY USING ARTIFICIAL INTELLIGENCE

(71) Applicants: THE CATHOLIC UNIVERSITY OF KOREA INDUSTRY-ACADEMIC COOPERATION FOUNDATION, Seoul (KR); GACHON UNIVERSITY OF INDUSTRY-ACADEMIC COOPERATION FOUNDATION, Seongnam-si, Gyeonggi-do (KR)

(72) Inventors: Sheen Woo LEE, Seoul (KR); Kwang Gi KIM, Seoul (KR)

(21) Appl. No.: 18/833,237

(22) PCT Filed: Jan. 12, 2023
(86) PCT No.: PCT/KR2023/000553

§ 371 Date: Jul. 25, 2024

(30) Foreign Application Priority Data
Jan. 26, 2022 (KR) 10-2022-0011605

(51) Int. Cl. A61B 6/03; A61B 6/50

(52) U.S. Cl. CPC A61B 6/032; A61B 6/505

(57) ABSTRACT

An embodiment provides a method for measuring a femoral anterior angle and a tibial torsion angle by using artificial intelligence, the method comprising the steps of: (a) obtaining, by a CT scanner, 2D CT images of the femur, the tibia, and the malleolus of a patient; (b) storing, in a big-data unit, the 2D CT images transmitted from the CT scanner; (c) setting, by an artificial intelligence unit, a first baseline related to the femoral head and neck, a second baseline related to the lower femoral, a third baseline related to the upper tibial, and a fourth baseline related to the malleolus, on the basis of the 2D CT images; and (d) measuring, by an angle measurement unit, a femoral anterior angle formed between the first baseline and the second baseline, and a tibial torsion angle formed between the third baseline and the fourth baseline.

### TECHNICAL FIELD

The present invention relates to a method for measuring a femoral anterior angle and a tibial torsion angle by using artificial intelligence, and more specifically, to a method for measuring a femoral anterior angle and a tibial torsion angle by using artificial intelligence, which automatically measures the femoral anterior angle and the tibial torsion angle using an artificial intelligence technology.

### BACKGROUND ART

The ankle joint is a joint that absorbs the shock applied to the foot and shank during walking of a human body and enables the human body to walk forward. In order to absorb the shock applied to the foot and shank when the foot contacts the ground during walking of a human body, the ankle joint must have sufficient flexibility. Accordingly, the ankle joint of a human body has a moving axis to respond to various spatial forms, and during the walking of the human body, the axis moves at an angle to an imaginary horizontal line when the foot in contact with the ground is viewed from above, or at an angle to the ground when the foot in contact with the ground is viewed from the side. This axis is also referred to as a “torsion axis.” In addition, various large and small bones that make up the ankle joint are firmly connected by connective tissue (e.g., ligaments) so that the ankle joint withstands the propulsive force applied to the foot and shank during walking of a human body.

Meanwhile, spasticity refers to an upper motor neuron syndrome caused by damage to the central nervous system, and is caused by hyperexcitability of the stretch reflex with velocity-dependent characteristics.

This spasticity phenomenon interferes with the walking of a human body. If the spasticity phenomenon persists, pain and deformation of the joint occur. In order to treat this spasticity phenomenon, an objective evaluation of the ankle joint is required. In the clinical field, the evaluation of the ankle joint is mainly performed by measuring the angle of the ankle joint. For example, the modified Tardieu scale (MTS) is an evaluation tool. However, since medical professionals in the clinical field currently mainly use a goniometer to measure the angle of the ankle joint, the measurement accuracy of the ankle joint is low, and devices that accurately measure the angle of the ankle joint are often very expensive commercially and difficult to operate.

Therefore, a device that is convenient for medical professionals to use in the clinical field and accurately measures the angle of the joint is required.

In response to the above requirement, there is an example of measuring the angle of the ankle joint using an inertial sensor. Since the ankle joint is short compared to other joints in a human body, the inertial sensor is small and light enough to be used for the ankle joint, and is portable and inexpensive. According to the method of measuring the angle of the ankle joint using an inertial sensor, having been disclosed so far, the angle of the ankle joint was measured without considering the torsional axis of the ankle joint, which changes during dorsiflexion and plantar flexion.

In addition, there was a problem that the inappropriate sensor position of the inertial sensor reduced the accuracy of the angle measurement of the ankle joint.

(Patent Document 1) Patent Publication No. 10-2019-0056647 (May 27, 2019)

### DETAILED DESCRIPTION OF THE INVENTION

### Technical Problem

An aspect of the present invention is to provide a method for measuring a femoral anterior angle and a tibial torsion angle by using artificial intelligence, which sets a first baseline related to the femoral head and neck, a second baseline related to the lower femur, a third baseline related to the upper part of the tibia, and a fourth baseline related to the malleolus using an artificial intelligence unit, and measures the femoral anterior angle formed by the first and second baselines and the tibial torsion angle formed by the third and fourth baselines.

The aspect of the present invention is not limited to that mentioned above, and other aspects not mentioned will be clearly understood by those skilled in the art from the description below.

### Technical Solution

To this end, the present invention provides a method for measuring a femoral anterior angle and a tibial torsion angle by using artificial intelligence, the method comprising the steps of: (a) obtaining, by a CT scanner. 2D CT images of the femur, the tibia, and the malleolus of a patient: (b) storing, in a big-data unit, the 2D CT images transmitted from the CT scanner; (c) setting, by an artificial intelligence unit, a first baseline related to the femoral head and neck, a second baseline related to the lower femur, a third baseline related to the upper tibia, and a fourth baseline related to the malleolus, based on the 2D CT images; and (d) measuring, by an angle measurement unit, a femoral anterior angle formed between the first baseline and the second baseline, and a tibial torsion angle formed between the third baseline and the fourth baseline.

In an embodiment of the present invention, the 2D CT images may include a plurality of femur CT images, a plurality of tibia CT images, and a plurality of malleolus CT images, and step (a) may include: (a1) a step in which the CT scanner scans the patient's femur from the top to the bottom of the patient's femur in the xy plane to obtain the plurality of femur CT images; (a2) a step in which the CT scanner scans the patient's tibia from the top to the bottom of the patient's tibia in the xy plane to obtain the plurality of tibia CT images; (a3) a step in which the CT scanner scans the patient's malleolus from the top to the bottom of the patient's malleolus in the xy plane to obtain the plurality of malleolus CT images; and (a4) a step in which the CT scanner transmits the plurality of femur CT images, the plurality of tibia CT images, and the plurality of malleolus CT images to the big-data unit.

In an embodiment of the present invention, step (a) may further include (a5) a step in which the CT scanner obtains 3D CT images of the femur, the tibia, and the malleolus of the patient based on the plurality of femur CT images, the plurality of tibia CT images, and the plurality of malleolus CT images using an artificial neural network.

In an embodiment of the present invention, step (b) may include: (b1) a step in which the big-data unit classifies and stores the plurality of femur CT images, the plurality of tibia CT images, and the plurality of malleolus CT images transmitted from the CT scanner, respectively; and (b2) a step in which the big-data unit transmits the plurality of femur CT images, the plurality of tibia CT images, and the plurality of malleolus CT images to the artificial intelligence unit.

In an embodiment of the present invention, step (c) may include: (c1) a step in which the artificial intelligence unit sets the first baseline for measuring a femoral anterior angle based on the plurality of femur CT images transmitted from the big-data unit; (c2) a step in which the artificial intelligence unit sets the second baseline for measuring a femoral anterior angle based on the plurality of femur CT images transmitted from the big-data unit; (c3) a step in which the artificial intelligence unit sets the third baseline for measuring a tibial torsion angle based on the plurality of tibia CT images transmitted from the big-data unit; and (c4) a step in which the artificial intelligence unit sets the fourth baseline for measuring a tibial torsion angle based on the plurality of malleolus CT images transmitted from the big-data unit.

In an embodiment of the present invention, step (c1) may include: (c11) a step in which the artificial intelligence unit receives a plurality of femoral head CT images from among the plurality of femur CT images transmitted from the big-data unit; (c12) a step in which the artificial intelligence unit finds a femoral head CT image in which the largest femoral head is captured among the plurality of femoral head CT images based on pre-learned femur CT images; and (c13) a step in which the artificial intelligence unit sets a first reference circle that contacts the largest femoral head in the femoral head CT image in which the largest femoral head is captured and the center of the first reference circle.

In an embodiment of the present invention, step (c) may further include: (c14) a step in which the artificial intelligence unit finds a femur CT image in which the largest femoral neck is captured among the plurality of femur CT images based on the pre-learned femur CT images: (c15) a step in which the artificial intelligence unit sets a first reference rectangle that contacts the largest femoral neck in the femur CT image in which the largest femoral neck is captured; and (c16) a step in which the artificial intelligence unit sets the first baseline parallel to the long axis of the first reference rectangle from the center of the first reference circle.

In an embodiment of the present invention, step (c2) may include: (c21) a step in which the artificial intelligence unit receives a plurality of lower femur CT images among the plurality of femur CT images transmitted from the big-data unit; (c22) a step in which the artificial intelligence unit finds a lower femur CT image in which the largest lower femur is captured among the plurality of lower femur CT images based on pre-learned femur CT images; and (c23) a step in which the artificial intelligence unit sets a second reference rectangle that contacts the largest lower femur in the lower femur CT image in which the largest lower femur is captured, and a first contact point in which the second reference rectangle contacts the largest lower femur.

In an embodiment of the present invention, step (c2) may further include: (c24) a step in which the artificial intelligence unit sets a plurality of first diagonal lines that connect from a vertex located on the other side of the lower part among the vertices of the second reference rectangle to the edge of the largest lower femur; (c25) a step in which the artificial intelligence unit sets a second contact point in which the shortest first diagonal line among the plurality of first diagonal lines contacts the largest lower femur; and (c26) a step in which the artificial intelligence unit sets the second baseline by connecting the first contact point and the second contact point.

In an embodiment of the present invention, step (c3) may include: (c31) a step in which the artificial intelligence unit receives a plurality of upper tibia CT images among the plurality of tibia CT images transmitted from the big-data unit; (c32) a step in which the artificial intelligence unit finds an upper tibia CT image in which the largest upper tibia is captured among the plurality of upper tibia CT images based on pre-learned tibia CT images; and (c33) a step in which the artificial intelligence unit sets a third reference rectangle that contacts the largest upper tibia in the upper tibia CT image in which the largest upper tibia is captured and a third contact point in which the third reference rectangle contacts the largest upper tibia.

In an embodiment of the present invention, step (c3) may further include: (c34) a step in which the artificial intelligence unit sets a plurality of second diagonal lines that connect from a vertex located on the other side of the lower part among the vertices of the third reference rectangle to the edge of the largest upper tibia; (c35) a step in which the artificial intelligence unit sets a fourth contact point in which the shortest second diagonal line among the plurality of second diagonal lines contacts the largest upper tibia; and (c36) a step in which the artificial intelligence unit sets the third baseline by connecting the third contact point and the fourth contact point.

In an embodiment of the present invention, step (c4) may include: (c41) a step in which the artificial intelligence unit receives the plurality of malleolus CT images transmitted from the big-data unit; (c42) a step in which the artificial intelligence unit finds a malleolus CT image in which the largest malleolus is captured among the plurality of malleolus CT images based on pre-learned malleolus CT images; (c43) a step in which the artificial intelligence unit sets a fourth reference rectangle that contacts the largest malleolus in the malleolus CT image in which the largest malleolus is captured; and (c44) a step in which the artificial intelligence unit sets a fourth baseline that bisects the fourth reference rectangle while being parallel to the long axis of the fourth reference rectangle.

### Advantageous Effects

The effect of the present invention according to the above configuration is that by using an artificial intelligence unit, a first baseline related to the femoral head and neck, a second baseline related to the lower femur, a third baseline related to the upper part of the tibia, and a fourth baseline related to the malleolus are set, and a femoral anterior angle formed by the first and second baselines and a tibial torsion angle formed by the third and fourth baselines are measured, so that it is possible to apply the present invention in a customized manner to the femur, the tibia, and the malleolus of various human bodies, and improve the measurement accuracy of the femoral anterior angle and the tibial torsion angle measured with a uniform standard.

The effects of the present invention are not limited to the effects described above, and should be understood to include all effects that are inferable from the configuration of the present invention described in the detailed description or claims of the present invention.

### BRIEF DESCRIPTION OF THE DRAWINGS

FIG. 1 is a flowchart showing a method for measuring a femoral anterior angle and a tibial torsion angle by using artificial intelligence according to an embodiment of the present invention.

(a) and (b) of FIG. 2 are each a view showing that a CT scanner used in a method for measuring a femoral anterior angle and a tibial torsion angle by using artificial intelligence according to an embodiment of the present invention implements a 3D CT image of the femur, the tibia, and the malleolus of a patient based on a plurality of femur CT images, a plurality of tibia CT images, and a plurality of malleolus CT images using an artificial neural network.

FIG. 3 is a view showing a method for measuring a femoral anterior angle and a tibial torsion angle using artificial intelligence according to an embodiment of the present invention, dividing the lower part of a human body (from the waist to the toes) to measure a femoral anterior angle and a tibial torsion angle.

(a), (b), and (c) of FIG. 4 each show a CT image of the femoral neck (femur neck) captured by a CT scanner in a method for measuring a femoral anterior angle and a tibial torsion angle by using artificial intelligence according to an embodiment of the present invention.

(a), (b), and (c) of FIG. 5 and FIG. 6 are each a view showing a process of setting a first baseline based on each patient's femur CT image in a method for measuring a femoral anterior angle and a tibial torsion angle by using artificial intelligence according to an embodiment of the present invention.

FIG. 7 is a femur CT image showing a first baseline set by a method for measuring a femoral anterior angle and a tibial torsion angle by using artificial intelligence according to an embodiment of the present invention.

FIG. 8 and FIG. 9 are each a view showing a process of setting a second baseline based on a lower femur CT image in a method for measuring a femoral anterior angle and a tibial torsion angle by using artificial intelligence according to an embodiment of the present invention.

(a), (b), (c), (d), (e), and (f) of FIG. 10 through FIG. 12 are each a view showing a first baseline or a second baseline according to the left or right side of each patient.

FIG. 13 is a lower femur CT image showing a second baseline set by a method for measuring a femoral anterior angle and a tibial torsion angle by using artificial intelligence according to an embodiment of the present invention.

FIG. 14 is a view showing a process of setting a third baseline based on an upper tibia CT image in a method for measuring a femoral anterior angle and a tibial torsion angle using artificial intelligence according to an embodiment of the present invention.

FIG. 15 is an upper tibia CT image showing a third baseline set by a method for measuring a femoral anterior angle and a tibial torsion angle by using artificial intelligence according to an embodiment of the present invention.

FIG. 16 and FIG. 17 are each a view showing a process of setting a fourth baseline based on a malleolus CT image in a method for measuring a femoral anterior angle and a tibial torsion angle by using artificial intelligence according to an embodiment of the present invention.

FIG. 18 is a malleolus CT image showing a fourth baseline set by a method for measuring a femoral anterior angle and a tibial torsion angle by using artificial intelligence according to an embodiment of the present invention.

FIG. 19 is a flowchart showing a method for setting a fourth baseline in a method for measuring a femoral anterior angle and a tibial torsion angle by using artificial intelligence according to an embodiment of the present invention.

(a) and (b) of FIG. 20 are each a view for explaining the coordinates of the inner malleolus, tibia, and outer malleolus and the internal angles of a triangle connecting the centers of gravity of the inner malleolus, tibia, and outer malleolus.

### BEST MODE FOR CARRYING OUT THE INVENTION

The most preferred embodiment according to the present invention includes the steps of: (a) obtaining, by a CT scanner, 2D CT images of the femur, the tibia, and the malleolus of a patient; (b) storing, in a big-data unit, the 2D CT images transmitted from the CT scanner; (c) setting, by an artificial intelligence unit, a first baseline related to the femoral head and neck, a second baseline related to the lower femur, a third baseline related to the upper tibia, and a fourth baseline related to the malleolus, based on the 2D CT images; and (d) measuring, by an angle measurement unit, a femoral anterior angle formed between the first baseline and the second baseline, and a tibial torsion angle formed between the third baseline and the fourth baseline.

### Mode for Carrying Out the Invention

Hereinafter, the present invention will be described with reference to the accompanying drawings.

However, the present invention may be implemented in various different forms and, therefore, is not limited to the examples described herein. In order to clearly explain the present invention in the drawings, portions unrelated to the description are omitted, and similar portions are given similar reference numerals throughout the specification.

Throughout the specification, when a portion is said to be “connected (linked, contacted, combined)” with another portion, this includes not only a case of being “directly connected” but also a case of being “indirectly connected” with another member in between. In addition, when a portion is said to “include” a certain component, this does not mean that other components are excluded, but that other components may be added, unless specifically stated to the contrary.

The terms used herein are merely used to describe specific embodiments and are not intended to limit the present invention. Singular expressions include plural expressions unless the context clearly dictates otherwise. In this specification, it should be understood terms such as “include” or “have” are to designate the presence of features, numbers, steps, operations, components, parts, or combinations thereof described in the specification, but are not to exclude in advance the possibility of the presence or addition of one or more other features, numbers, steps, operations, components, parts, or combinations thereof.

Hereinafter, embodiments of the present invention will be described in detail with reference to the accompanying drawings.

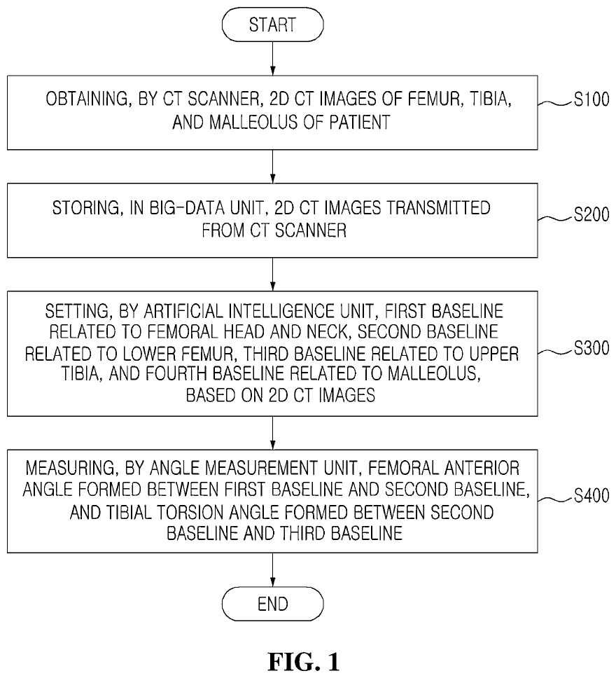

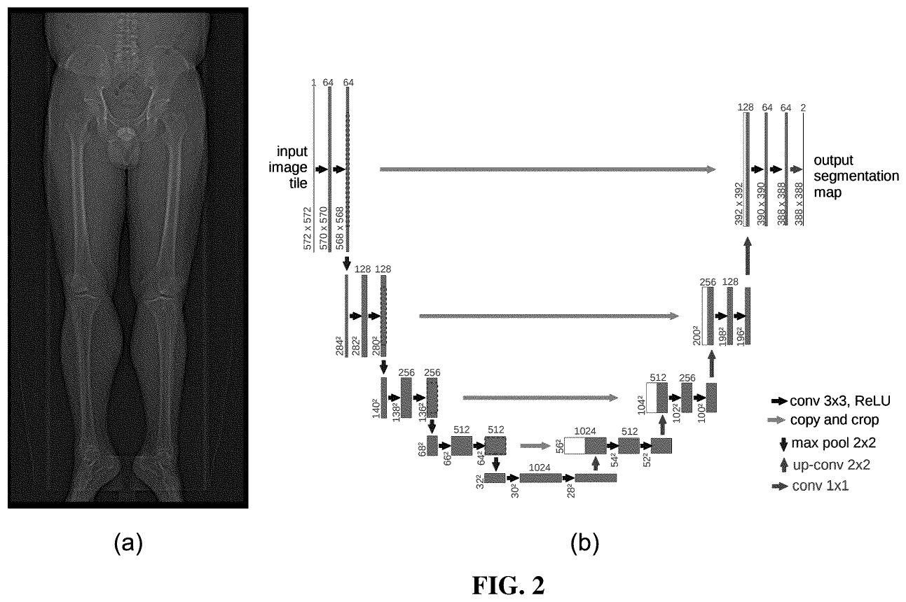

FIG. 1 is a flowchart showing a method for measuring a femoral anterior angle and a tibial torsion angle by using artificial intelligence according to an embodiment of the present invention.

Referring to FIG. 1, a method for measuring a femoral anterior angle and a tibial torsion angle by using artificial intelligence according to an embodiment of the present invention includes the steps of: (a) obtaining, by a CT scanner, 2D CT images of the femur, the tibia, and the malleolus of a patient (S100); (b) storing, in a big-data unit, the 2D CT images transmitted from the CT scanner (S200); (c) setting, by an artificial intelligence unit, a first baseline related to the femoral head and neck, a second baseline related to the lower femur, a third baseline related to the upper tibia, and a fourth baseline related to the malleolus, based on the 2D CT images (S300); and (d) measuring, by an angle measurement unit, a femoral anterior angle formed between the first baseline and the second baseline, and a tibial torsion angle formed between the third baseline and the fourth baseline.

First, step (a) includes: (a1) a step in which the CT scanner scans the patient's femur from the top to the bottom of the patient's femur in the xy plane to obtain the plurality of femur CT images; (a2) a step in which the CT scanner scans the patient's tibia from the top to the bottom of the patient's tibia in the xy plane to obtain the plurality of tibia CT images; (a3) a step in which the CT scanner scans the patient's malleolus from the top to the bottom of the patient's malleolus in the xy plane to obtain the plurality of malleolus CT images; and (a4) a step in which the CT scanner transmits the plurality of femur CT images, the plurality of tibia CT images, and the plurality of malleolus CT images to the big-data unit.

In step (a), the CT scanner scans, in 2D, the body of the patient standing or lying down. At this time, the scanned image is a 2D CT image sliced in the xy plane when the patient is standing.

FIGS. 2A and 2B are each a view showing that a CT scanner used in a method for measuring a femoral anterior angle and a tibial torsion angle by using artificial intelligence according to an embodiment of the present invention implements a 3D CT image of the femur, the tibia, and the malleolus of a patient based on a plurality of femur CT images, a plurality of tibia CT images, and a plurality of malleolus CT images using an artificial neural network.

In addition, step (a) further includes (a5) a step in which the CT scanner obtains 3D CT images of the femur, the tibia, and the malleolus of the patient based on the plurality of femur CT images, the plurality of tibia CT images, and the plurality of malleolus CT images using an artificial neural network.

Specifically, in step (a5), the CT scanner uses U-net, one of the artificial neural networks, as shown in FIG. 2B, to implement a 3D CT image based on a 2D CT image.

Accordingly, the CT scanner implements a 3D CT image based on a 2D CT image using an artificial neural network, as shown in FIG. 2A.

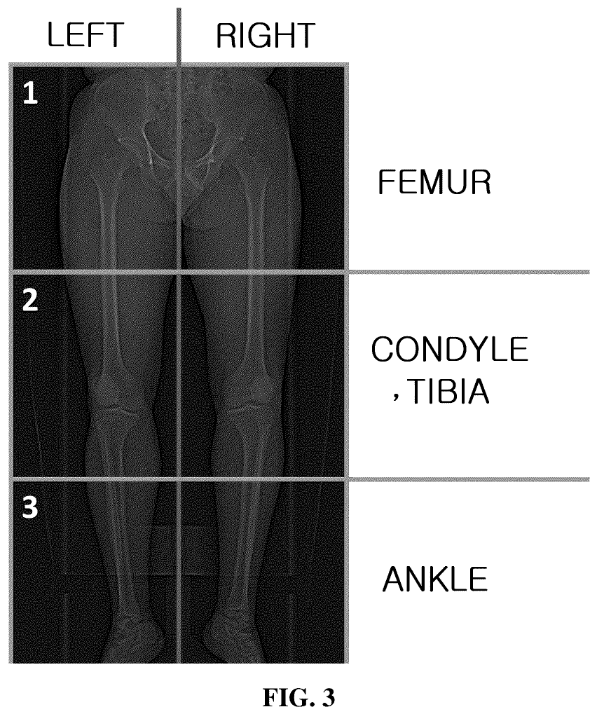

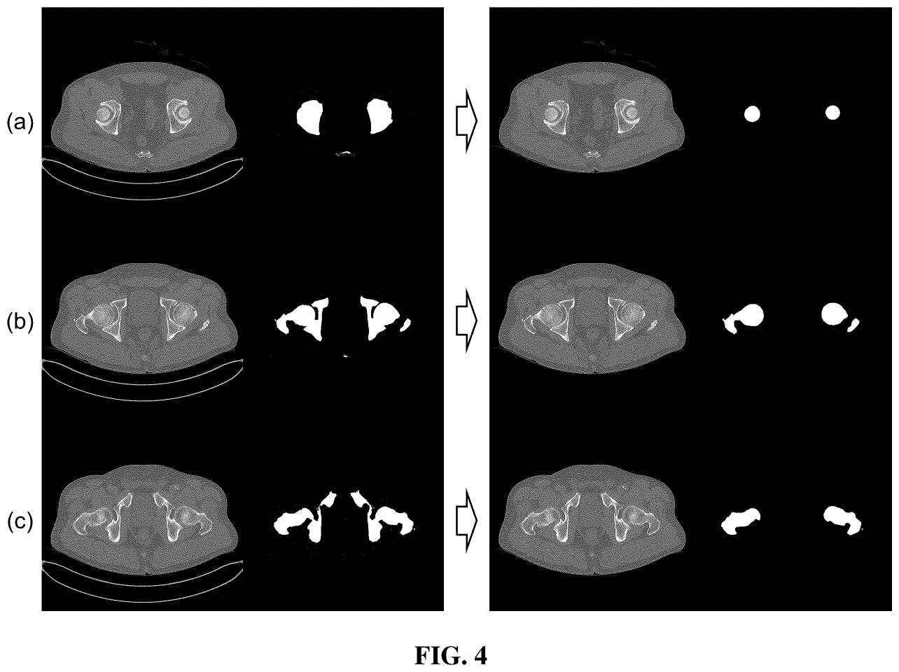

FIG. 3 is a view showing a method for measuring a femoral anterior angle and a tibial torsion angle using artificial intelligence according to an embodiment of the present invention, dividing the lower part of a human body (from the waist to the toes) to measure a femoral anterior angle and a tibial torsion angle.

Referring to FIG. 3, in step (a), the 2D CT images include a plurality of femur CT images, a plurality of tibia CT images, and a plurality of malleolus CT images.

Next, step (b) includes: (b1) a step in which the big-data unit classifies and stores the plurality of femur CT images, the plurality of tibia CT images, and the plurality of malleolus CT images transmitted from the CT scanner, respectively; and (b2) a step in which the big-data unit transmits the plurality of femur CT images, the plurality of tibia CT images, and the plurality of malleolus CT images to the artificial intelligence unit.

Next, step (c) includes: (c1) a step in which the artificial intelligence unit sets the first baseline for measuring a femoral anterior angle based on the plurality of femur CT images transmitted from the big-data unit; (c2) a step in which the artificial intelligence unit sets the second baseline for measuring a femoral anterior angle based on the plurality of femur CT images transmitted from the big-data unit; (c3) a step in which the artificial intelligence unit sets the third baseline for measuring a tibial torsion angle based on the plurality of tibia CT images transmitted from the big-data unit; and (c4) a step in which the artificial intelligence unit sets the fourth baseline for measuring a tibial torsion angle based on the plurality of malleolus CT images transmitted from the big-data unit.

FIGS. 4A, 4B, and 4C each show a CT image of the femoral neck (femur neck) captured by a CT scanner in a method for measuring a femoral anterior angle and a tibial torsion angle by using artificial intelligence according to an embodiment of the present invention.

Specifically, step (c1) includes: (c11) a step in which the artificial intelligence unit receives a plurality of femoral head CT images from among the plurality of femur CT images transmitted from the big-data unit; (c12) a step in which the artificial intelligence unit finds a femoral head CT image in which the largest femoral head is captured among the plurality of femoral head CT images based on pre-learned femur CT images; and (c13) a step in which the artificial intelligence unit sets a first reference circle C1 that contacts the largest femoral head in the femoral head CT image in which the largest femoral head is captured and the center CP1 of the first reference circle C1.

At this time, the pre-learned femur CT images include a criterion for distinguishing the femoral head and femoral neck by learning of 2D CT images of the femurs of many people by a deep learning unit.

That is, the deep learning unit distinguishes to which part of input femur CT images is the femoral head or the femoral neck based on the pre-learned femur CT images, and finds a femur CT image with the largest femoral head captured and a femur CT image with the largest femoral neck captured, based on the pre-learned femur CT images.

In addition, pre-learned lower femur CT images, pre-learned tibia CT images, and pre-learned malleolus CT images, which will be described later, are also utilized through the deep learning unit.

The left side of each of FIGS. 4A, 4B, and 4C illustrates a femoral head CT image according to a change in position along the z-axis, and the right side of each of FIGS. 4A, 4B, and 4C illustrates image processing of the femoral head CT image shown on the left side of each of FIGS. 4A, 4B, and 4C.

Specifically, FIGS. 4A and 4B are images processed by focusing on the femoral head, and FIG. 4C is an image processed by focusing on the femoral neck.

In step (c12), from among a plurality of femoral head CT images according to a change in position along the z-axis, the femoral head CT image in which the largest femoral head is captured, as shown in FIG. 4B, is found.

FIGS. 5A, 5B, and 5C and FIGS. 6A, 6B, and 6C are each a view showing a process of setting a first baseline based on each patient's femur CT image in a method for measuring a femoral anterior angle and a tibial torsion angle by using artificial intelligence according to an embodiment of the present invention.

Next, in step (c13), the first reference circle CP1 that contacts the largest femoral head is set in the femoral head CT image in which the largest femoral head is captured, as shown in FIG. 5A and FIG. 6A.

In addition, step (c) further includes: (c14) a step in which the artificial intelligence unit finds a femur CT image in which the largest femoral neck is captured among the plurality of femur CT images based on the pre-learned femur CT images; (c15) a step in which the artificial intelligence unit sets a first reference rectangle Q1 that contacts the largest femoral neck in the femur CT image in which the largest femoral neck is captured; and (c16) a step in which the artificial intelligence unit sets the first baseline L1 parallel to the long axis of the first reference rectangle C1 from the center of the first reference circle C1.

In step (c15), the artificial intelligence unit sets the first reference rectangle Q1 that contacts the largest femoral neck in the femur CT image in which the largest femoral neck is captured, as shown in FIG. 5B and FIG. 5B.

Next, in step (c16), the artificial intelligence unit sets the first baseline L1 parallel to the long axis of the first reference rectangle C1 from the center of the first reference circle C1, as shown in FIG. 5C and FIG. 6C.

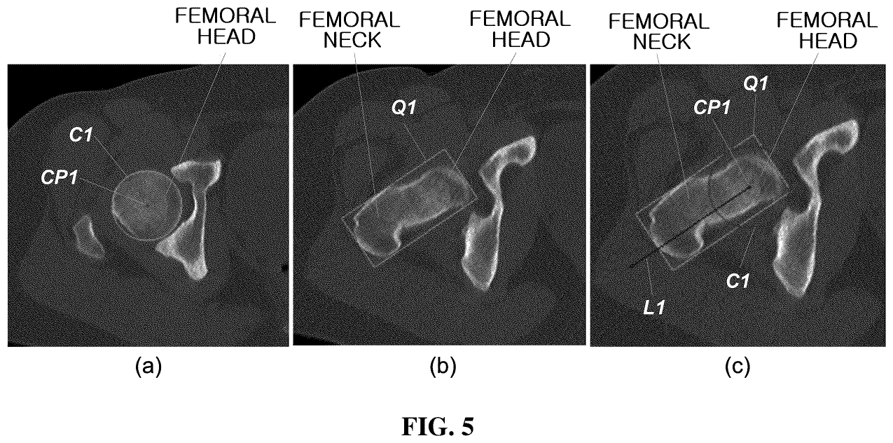

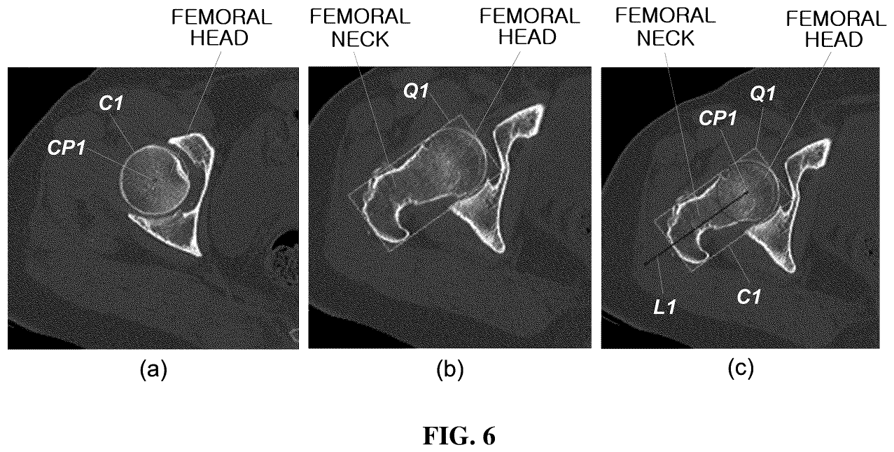

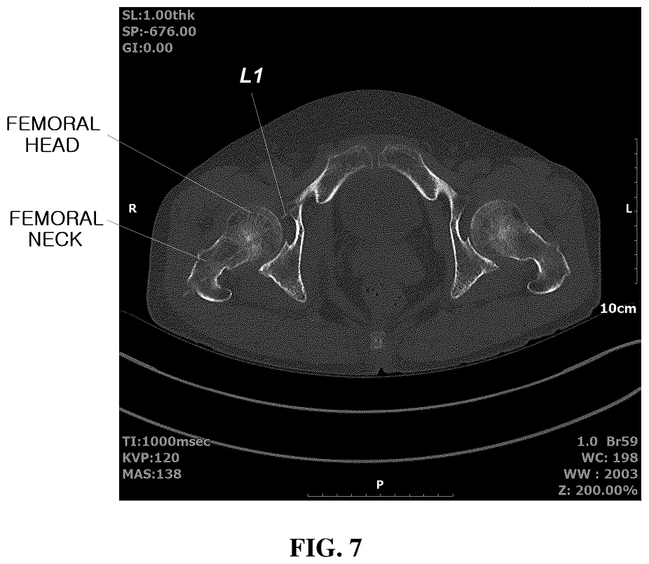

FIG. 7 is a femur CT image showing a first baseline set by a method for measuring a femoral anterior angle and a tibial torsion angle by using artificial intelligence according to an embodiment of the present invention.

Through steps (c11) to (c16), the first baseline L1 is set as shown in FIG. 7.

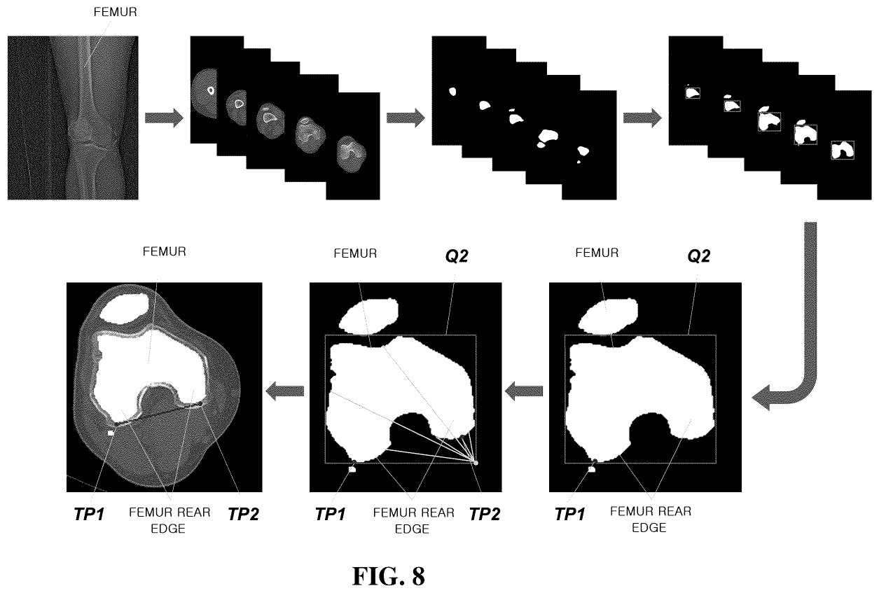

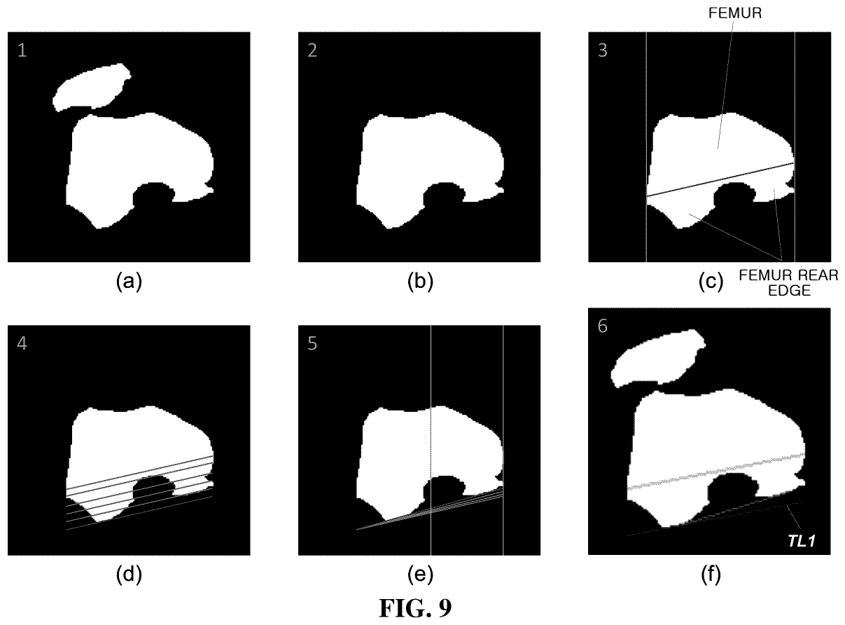

FIG. 8 and FIGS. 9A, 9B, 9C, 9D, 9E, and 9F are each a view showing a process of setting a second baseline based on a lower femur CT image in a method for measuring a femoral anterior angle and a tibial torsion angle by using artificial intelligence according to an embodiment of the present invention.

Referring to FIG. 8, (c2) includes: (c21) a step in which the artificial intelligence unit receives a plurality of lower femur CT images among the plurality of femur CT images transmitted from the big-data unit; (c22) a step in which the artificial intelligence unit finds a lower femur CT image in which the largest lower femur is captured among the plurality of lower femur CT images based on pre-learned femur CT images; and (c23) a step in which the artificial intelligence unit sets a second reference rectangle Q2 that contacts the largest lower femur in the lower femur CT image in which the largest lower femur is captured, and a first contact point TL1 in which the second reference rectangle Q2 contacts the largest lower femur.

In step (c21), the artificial intelligence unit receives the plurality of lower femur CT images among the plurality of femur CT images shown in FIG. 8 (the second and third views from the left of the upper part of FIG. 8).

Next, in step (c22), the artificial intelligence unit finds the lower femur CT image in which the largest lower femur is captured among the plurality of lower femur CT images shown in FIG. 8 (the view shown on the far right of the upper part of FIG. 8).

Next, in step (c23), as shown in FIG. 8 (the view shown on the far right of the lower part of FIG. 8), the second reference rectangle Q2 that contacts the largest lower femur in the lower femur CT image in which the largest lower femur is captured and the first contact point TP1 in which the second reference rectangle Q2 contacts the largest lower femur are set.

Next, referring to FIG. 8, step (c2) further includes: (c24) a step in which the artificial intelligence unit sets a plurality of first diagonal lines (shown in yellowish green in FIG. 8) that connect from a vertex located on the other side of the lower part among the vertices of the second reference rectangle Q2 to the edge of the largest lower femur; (c25) a step in which the artificial intelligence unit sets a second contact point TP2 in which the shortest first diagonal line among the plurality of first diagonal lines contacts the largest lower femur; and (c26) a step in which the artificial intelligence unit sets the second baseline TL2 by connecting the first contact point TP1 and the second contact point TP2.

In step (c24), as shown in FIG. 8 (the view shown in the center of the lower part of FIG. 8), a plurality of first diagonal lines are set that connect from a vertex located on the other side of the lower part among the vertices of the second reference rectangle Q2 to the edge of the largest lower femur.

Next, in step (c25), as shown in FIG. 8 (the view shown in the center of the lower part of FIG. 8), the second contact point TP2 in which the shortest first diagonal line among the plurality of first diagonal lines contacts the largest lower femur is set.

Next, in step (c26), as shown in FIG. 8 (the view shown in the far left of the lower part of FIG. 8), the second baseline TL2 is set by connecting the first contact point TP1 and the second contact point TP2.

Meanwhile, in addition to the method shown in FIG. 8, the second baseline may also be set using the method shown in FIGS. 9A, 9B, 9C, 9D, 9E, and 9F.

Specifically, referring to FIGS. 9A, 9B, 9C, 9D, 9E, and 9F, two extension lines that extend in the upper and lower directions while contacting the femur are set in the lower femur CT image, and then a connecting line (the view shown in the far right of the upper part of FIG. 9) that connects a contact point in which the two extension lines contact the femur is set.

Next, a plurality of parallel lines parallel to the connecting line (the view shown on the far left of the lower part of FIG. 9) are set, and then the angles of the parallel lines (the view shown on the center of the lower part of FIG. 9) are changed to set the second baseline TL2 (the view shown on the far right of the lower part of FIG. 9).

FIGS. 10A, 10B, 10C, 10D, 10E, and 10F, FIGS. 11A, 11B, 11C, 11D, 11E, and 11F, and FIGS. 12A, 12B, 12C, 12D, 12E, and 12F are each a view showing a first baseline or a second baseline according to the left or right side of each patient.

The first baseline L1 and second baseline TL1 set as above are set to measure the femoral anterior angle a as shown in FIGS. 10A, 10B, and 10C, FIGS. 10D, 10E, and 10F, FIGS. 11A, 11B, and 11C, FIGS. 11D, 11E, and 11F, FIGS. 12A, 12B, and 12C, and FIGS. 12D, 12E, and 12F.

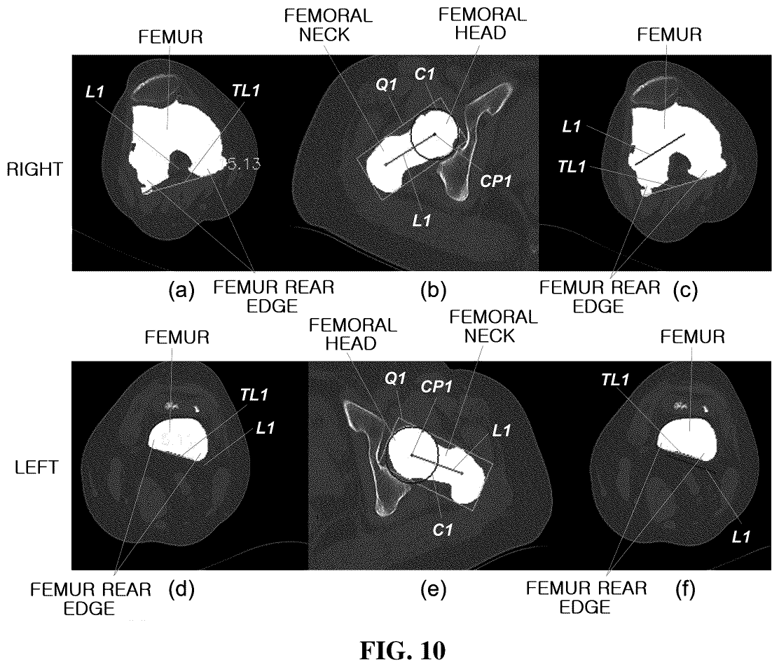

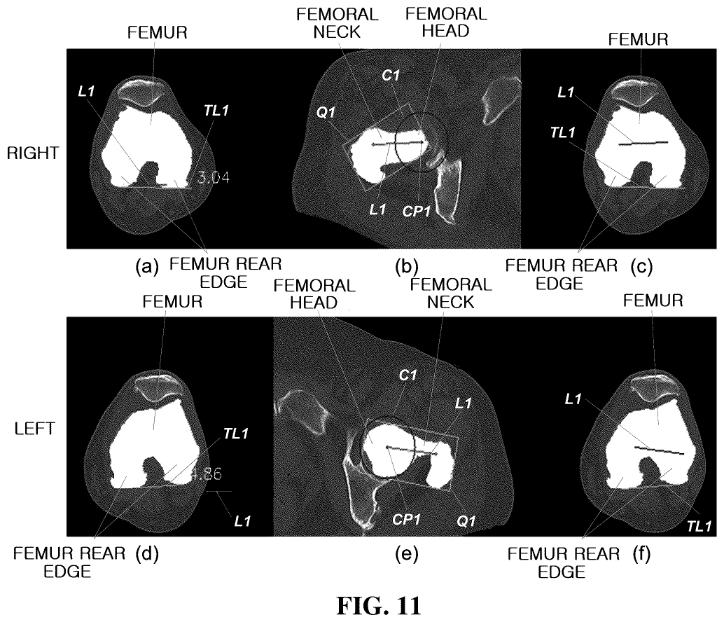

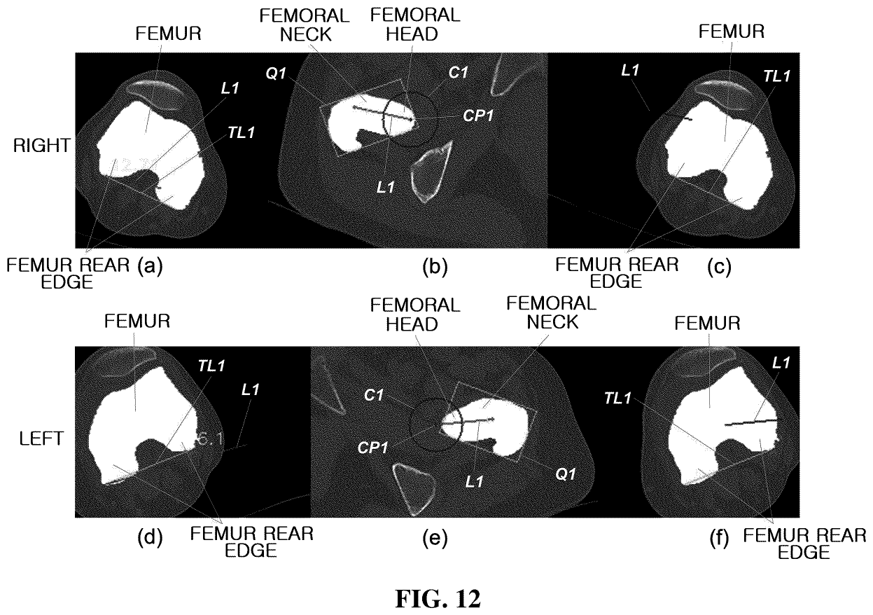

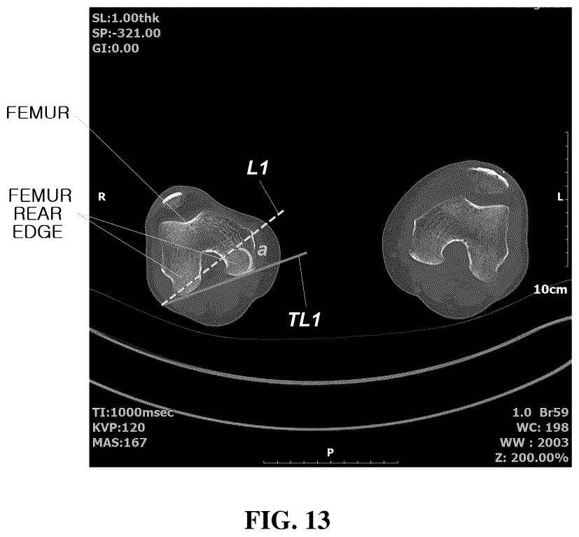

FIG. 13 is a lower femur CT image showing a second baseline set by a method for measuring a femoral anterior angle and a tibial torsion angle by using artificial intelligence according to an embodiment of the present invention.

The femoral anterior angle a formed by the first baseline L1 and second baseline TL1 described above is shown in FIG. 13.

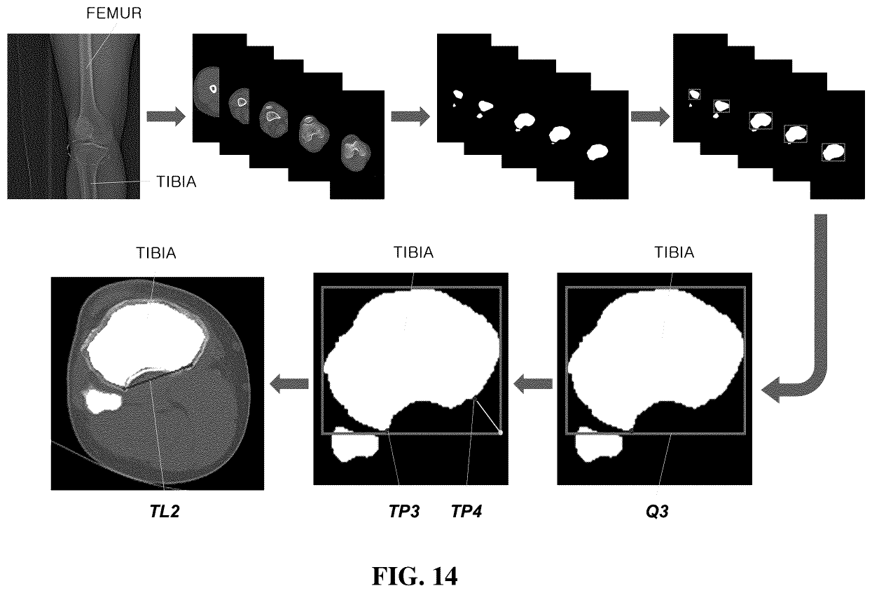

FIG. 14 is a view showing a process of setting a third baseline based on an upper tibia CT image in a method for measuring a femoral anterior angle and a tibial torsion angle using artificial intelligence according to an embodiment of the present invention.

Next, referring to FIG. 14, step (c3) includes: (c31) a step in which the artificial intelligence unit receives a plurality of upper tibia CT images among the plurality of tibia CT images transmitted from the big-data unit; (c32) a step in which the artificial intelligence unit finds an upper tibia CT image in which the largest upper tibia is captured among the plurality of upper tibia CT images based on pre-learned tibia CT images; and (c33) a step in which the artificial intelligence unit sets a third reference rectangle Q3 that contacts the largest upper tibia in the upper tibia CT image in which the largest upper tibia is captured and a third contact point TP3 in which the third reference rectangle Q3 contacts the largest upper tibia.

At this time, the pre-learned tibia CT images include a criterion for distinguishing the upper tibia by learning of 2D CT images of the tibia of many people by the deep learning unit.

That is, the deep learning unit distinguishes to which part of input tibia CT images is the upper tibia based on the pre-learned tibia CT images, and finds a tibia CT image with the largest upper tibia captured among the plurality of tibia CT images, based on the pre-learned tibia CT images.

In step (c31), the artificial intelligence unit receives the plurality of upper tibia CT images among the plurality of tibia CT images shown in FIG. 14 (the second and third vies from the left of the upper part of FIG. 14).

Next, in step (c32), the artificial intelligence unit finds the upper tibia CT image in which the largest upper tibia is captured among the plurality of upper tibia CT images shown in FIG. 14 (the view shown on the far right of the upper part of FIG. 14).

Next, in step (c33), as shown in FIG. 14 (the view shown on the far right of the lower part of FIG. 14), the third reference rectangle Q3 that contacts the largest upper tibia in the upper tibia CT image in which the largest upper tibia is captured and the third contact point TP3 in which the largest upper tibia contacts the largest upper tibia are set.

Next, referring to FIG. 14, step (c3) further includes: (c34) a step in which the artificial intelligence unit sets a plurality of second diagonal lines that connect from a vertex located on the other side of the lower part among the vertices of the third reference rectangle Q3 to the edge of the largest upper tibia; (c35) a step in which the artificial intelligence unit sets a fourth contact point TP4 in which the shortest second diagonal line among the plurality of second diagonal lines contacts the largest upper tibia; and (c36) a step in which the artificial intelligence unit sets the third baseline TL2 by connecting the third contact point TP3 and the fourth contact point TP4.

In step (c34), as shown in FIG. 14 (the view shown in the center of the lower part of FIG. 14), the plurality of second diagonal lines that connect from the vertex located on the other side of the lower part among the vertices of the third reference rectangle Q3 to the edge of the largest upper tibia are set.

Next, in step (c35), as shown in FIG. 14 (the view shown in the center of the lower part of FIG. 14), the fourth contact point TP4 in which the shortest second diagonal line among the plurality of second diagonal lines contacts the largest upper tibia is set.

Next, in step (c36), as shown in FIG. 14 (the view shown in the center of the lower part of FIG. 14), the third baseline TL2 is set by connecting the third contact point TP3 and the fourth contact point TP4.

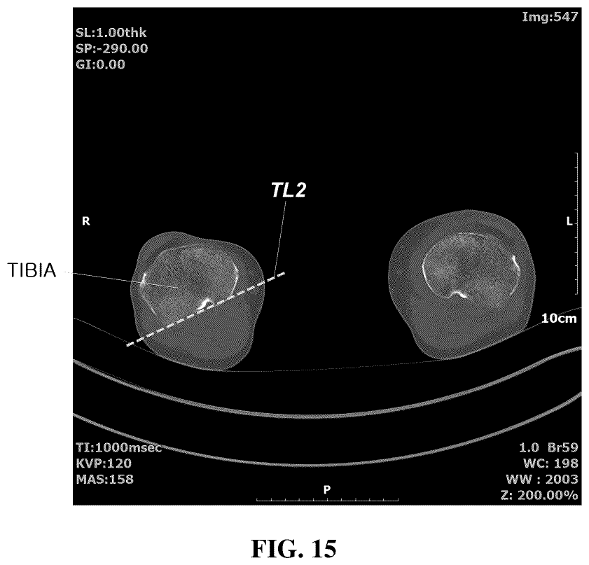

FIG. 15 is an upper tibia CT image showing a third baseline set by a method for measuring a femoral anterior angle and a tibial torsion angle by using artificial intelligence according to an embodiment of the present invention.

The third baseline TL2 set through the above process is shown in FIG. 15.

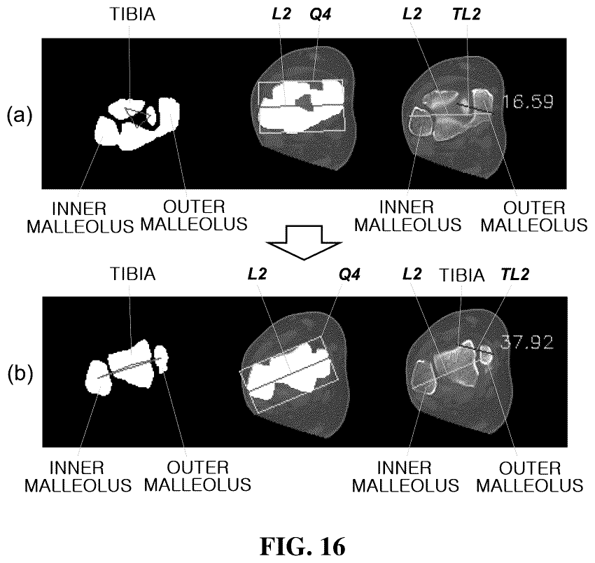

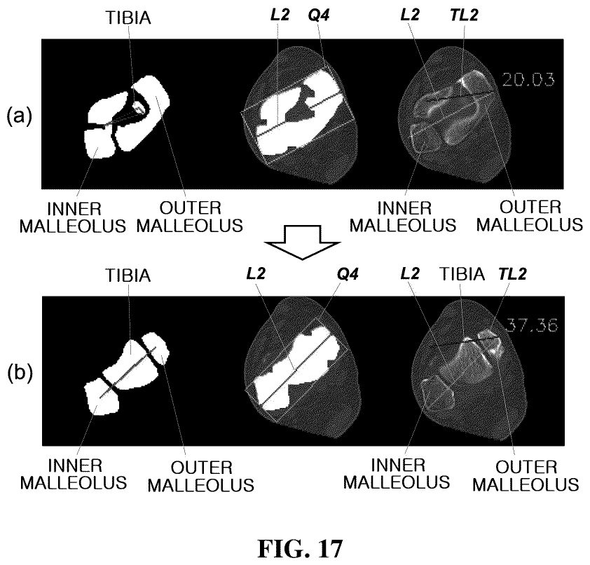

FIG. 16 and FIG. 17 are each a view showing a process of setting a fourth baseline based on a malleolus CT image in a method for measuring a femoral anterior angle and a tibial torsion angle by using artificial intelligence according to an embodiment of the present invention.

Next, referring to FIGS. 16 and 17, step (c4) includes: (c41) a step in which the artificial intelligence unit receives the plurality of malleolus CT images transmitted from the big-data unit; (c42) a step in which the artificial intelligence unit finds a malleolus CT image in which the largest malleolus is captured among the plurality of malleolus CT images based on pre-learned malleolus CT images; (c43) a step in which the artificial intelligence unit sets a fourth reference rectangle Q4 that contacts the largest malleolus in the malleolus CT image in which the largest malleolus is captured; and (c44) a step in which the artificial intelligence unit sets a fourth baseline L2 that bisects the fourth reference rectangle Q4 while being parallel to the long axis of the fourth reference rectangle Q4.

At this time, the pre-learned malleolus CT images include a criterion for distinguishing the malleolus by learning of 2D CT images of the malleolus of many people by the deep learning unit.

That is, the deep learning unit distinguishes to which part of input malleolus CT images is the malleolus based on the pre-learned femur CT images, and finds a malleolus CT image with the largest malleolus captured, based on the pre-learned malleolus CT images.

In step (c43), the fourth reference rectangle Q4 that contacts the largest malleolus in the malleolus CT image in which the largest malleolus is captured is set, as shown in (a) and (b) of FIG. 16 and (a) and (b) of FIG. 17.

Next, in step (c44), the fourth baseline L2 that is parallel to the long axis of the fourth reference rectangle Q4 and bisects the fourth reference rectangle Q4 is set, as shown in (a) and (b) of FIG. 16 and (a) and (b) of FIG. 17.

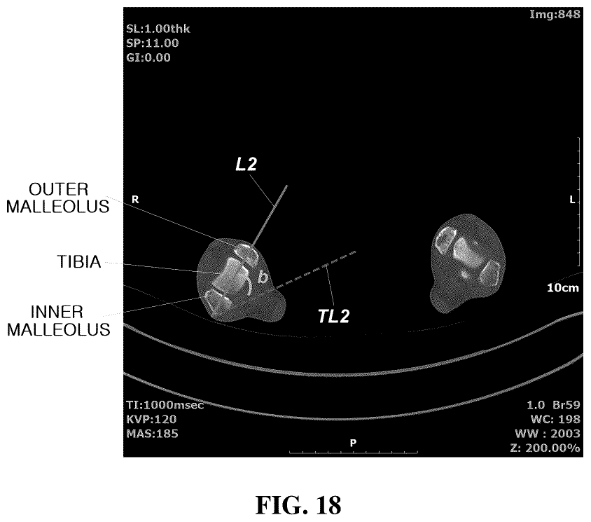

FIG. 18 is a malleolus CT image showing a fourth baseline set by a method for measuring a femoral anterior angle and a tibial torsion angle by using artificial intelligence according to an embodiment of the present invention.

The third baseline L2 set through steps (c31) to (c36), the fourth baseline TL2 set through steps (c41) to (c44), and the tibial torsion angle b formed by the third baseline L2 and the fourth baseline TL2 are shown in FIG. 18, and the angle measurement unit measures the tibial torsion angle b formed by the third baseline L2 and the fourth baseline TL2.

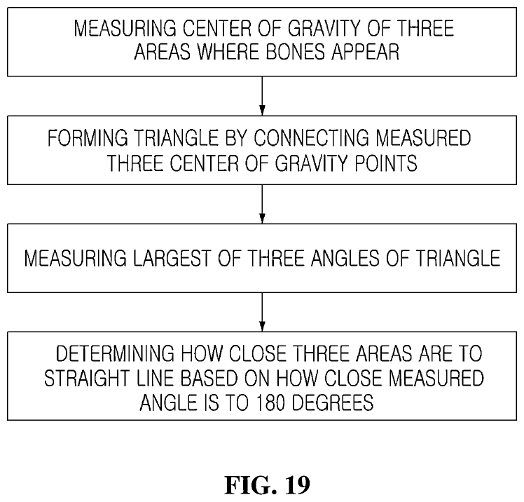

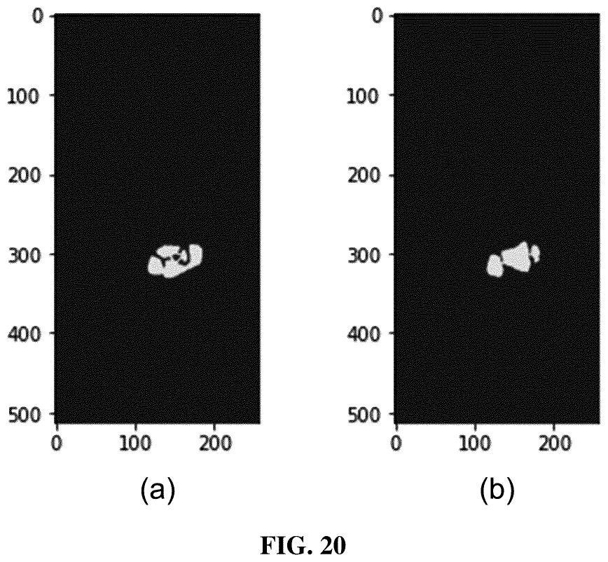

FIG. 19 is a flowchart showing a method for setting a fourth baseline in a method for measuring a femoral anterior angle and a tibial torsion angle by using artificial intelligence according to an embodiment of the present invention.

Referring to FIG. 19, the centers of gravity of three bones (inner malleolus, tibia, outer malleolus) are set, and the three set centers of gravity are connected to set a triangle shown in (a) and (d) of FIG. 16 and (a) and (d) of FIG. 17.

Next, the largest internal angle among the three internal angles of a triangle is measured.

Next, it is confirmed how close three areas are to a straight line based on how close the largest internal angle is to 180°.

FIGS. 20A and 20B are each a view for explaining the coordinates of the inner malleolus, tibia, and outer malleolus and the internal angles of a triangle connecting the centers of gravity of the inner malleolus, tibia, and outer malleolus.

In FIG. 20A, the three internal angles of a triangle are 58.22°, 42.05°, and 79.73°, respectively, and the coordinates for the three vertices of the triangle are (162, 303), (141, 297), and (151, 313), respectively.

In addition, in FIG. 20B, the three internal angles of a triangle are 4.03°, 5.15°, and 170.82°,respectively, and the coordinates for the three vertices of the triangle are (126, 316), (177, 300), and (154, 305), respectively.

Meanwhile, the present invention provides an apparatus for measuring a femoral anterior angle and a tibial torsion angle by using artificial intelligence, the apparatus being for implementing the above-described method for measuring a femoral anterior angle and a tibial torsion angle by using artificial intelligence.

The apparatus for measuring a femoral anterior angle and a tibial torsion angle by using artificial intelligence according to an embodiment of the present invention includes a CT scanner, a big-data unit, an artificial intelligence unit, and an angle measurement unit, and the descriptions above may be referred to for specific descriptions of the CT scanner, big-data unit, artificial intelligence unit, and angle measurement unit.

The description of the present invention described above is for illustrative purposes, and those skilled in the art will understand that the present invention is easily modifiable into other specific forms without changing the technical idea or essential features of the present invention. Therefore, the embodiments described above should be understood in all respects as illustrative and not restrictive. For example, each component described as single may be implemented in a distributed manner, and similarly, components described as distributed may also be implemented in a combined form.

The scope of the present invention is indicated by the claims described below, and all changes or modified forms derived from the meaning and scope of the claims and their equivalent concepts should be construed as being included in the scope of the present invention.

### EXPLANATION OF REFERENCE NUMERALS

C1: first reference circle
CP1: center of first reference circle
Q1: first reference rectangle
L1: first baseline
Q2: second reference rectangle
TP1: first contact point
TP2: second contact point
TL1: second baseline (first contact line)
Q3: third reference rectangle
TP3: third contact point
TP4: fourth contact point
TL2: third baseline (second contact line)
Q4: fourth reference rectangle
L2: fourth baseline

1. A method for measuring a femoral anterior angle and a tibial torsion angle by using artificial intelligence, the method comprising steps of:
(a) obtaining, by a CT scanner, 2D CT images of a femur, a tibia, and a malleolus of a patient;
(b) storing, in a big-data unit, the 2D CT images transmitted from the CT scanner;
(c) setting, by an artificial intelligence unit, a first baseline related to femoral head and neck, a second baseline related to a lower femur, a third baseline related to an upper tibia, and a fourth baseline related to the malleolus, based on the 2D CT images; and
(d) measuring, by an angle measurement unit, the femoral anterior angle formed between the first baseline and the second baseline, and the tibial torsion angle formed between the third baseline and the fourth baseline.

2. The method of claim 1, wherein the 2D CT images comprise a plurality of femur CT images, a plurality of tibia CT images, and a plurality of malleolus CT images, and the step (a) comprises:
(a1) a step in which the CT scanner scans the patient's femur from a top to a bottom of the patient's femur in a xy plane to obtain the plurality of femur CT images;
(a2) a step in which the CT scanner scans the patient's tibia from a top to a bottom of the patient's tibia in the xy plane to obtain the plurality of tibia CT images;
(a3) a step in which the CT scanner scans the patient's malleolus from a top to a bottom of the patient's malleolus in the xy plane to obtain the plurality of malleolus CT images; and
(a4) a step in which the CT scanner transmits the plurality of femur CT images, the plurality of tibia CT images, and the plurality of malleolus CT images to the big-data unit.

3. The method of claim 2, wherein the step (a) further comprises (a5) a step in which the CT scanner obtains 3D CT images of the femur, the tibia, and the malleolus of the patient based on the plurality of femur CT images, the plurality of tibia CT images, and the plurality of malleolus CT images using an artificial neural network.

4. The method of claim 2, wherein the step (b) comprises:
(b1) a step in which the big-data unit classifies and stores the plurality of femur CT images, the plurality of tibia CT images, and the plurality of malleolus CT images transmitted from the CT scanner, respectively; and
(b2) a step in which the big-data unit transmits the plurality of femur CT images, the plurality of tibia CT images, and the plurality of malleolus CT images to the artificial intelligence unit.

5. The method of claim 4, wherein the step (c) comprises:
(c1) a step in which the artificial intelligence unit sets the first baseline for measuring a femoral anterior angle based on the plurality of femur CT images transmitted from the big-data unit;
(c2) a step in which the artificial intelligence unit sets the second baseline for measuring a femoral anterior angle based on the plurality of femur CT images transmitted from the big-data unit;
(c3) a step in which the artificial intelligence unit sets the third baseline for measuring a tibial torsion angle based on the plurality of tibia CT images transmitted from the big-data unit; and
(c4) a step in which the artificial intelligence unit sets the fourth baseline for measuring a tibial torsion angle based on the plurality of malleolus CT images transmitted from the big-data unit.

6. The method of claim 5, wherein the step (c1) comprises:
(c11) a step in which the artificial intelligence unit receives a plurality of femoral head CT images from among the plurality of femur CT images transmitted from the big-data unit;
(c12) a step in which the artificial intelligence unit finds a femoral head CT image in which the largest femoral head is captured among the plurality of femoral head CT images based on pre-learned femur CT images; and
(c13) a step in which the artificial intelligence unit sets a first reference circle that contacts the largest femoral head in the femoral head CT image in which the largest femoral head is captured and the center of the first reference circle.

7. The method of claim 6, wherein the step (c) further comprises:
(c14) a step in which the artificial intelligence unit finds a femur CT image in which the largest femoral neck is captured among the plurality of femur CT images based on the pre-learned femur CT images;
(c15) a step in which the artificial intelligence unit sets a first reference rectangle that contacts the largest femoral neck in the femur CT image in which the largest femoral neck is captured; and
(c16) a step in which the artificial intelligence unit sets the first baseline parallel to a long axis of the first reference rectangle from the center of the first reference circle.

8. The method of claim 5, wherein the step (c2) comprises:
(c21) a step in which the artificial intelligence unit receives a plurality of lower femur CT images among the plurality of femur CT images transmitted from the big-data unit;
(c22) a step in which the artificial intelligence unit finds a lower femur CT image in which the largest lower femur is captured among the plurality of lower femur CT images based on pre-learned femur CT images; and
(c23) a step in which the artificial intelligence unit sets a second reference rectangle that contacts the largest lower femur in the lower femur CT image in which the largest lower femur is captured, and a first contact point in which the second reference rectangle contacts the largest lower femur.

9. The method of claim 6, wherein the step (c2) further comprises:
(c24) a step in which the artificial intelligence unit sets a plurality of first diagonal lines that connect from a vertex located on another side of a lower part among vertices of the second reference rectangle to an edge of the largest lower femur;
(c25) a step in which the artificial intelligence unit sets a second contact point in which the shortest first diagonal line among the plurality of first diagonal lines contacts the largest lower femur; and
(c26) a step in which the artificial intelligence unit sets the second baseline by connecting the first contact point and the second contact point.

10. The method of claim 5, wherein the step (c3) comprises:
(c31) a step in which the artificial intelligence unit receives a plurality of upper tibia CT images among the plurality of tibia CT images transmitted from the big-data unit;
(c32) a step in which the artificial intelligence unit finds an upper tibia CT image in which the largest upper tibia is captured among the plurality of upper tibia CT images based on pre-learned tibia CT images; and
(c33) a step in which the artificial intelligence unit sets a third reference rectangle that contacts the largest upper tibia in the upper tibia CT image in which the largest upper tibia is captured and a third contact point in which the third reference rectangle contacts the largest upper tibia.

11. The method of claim 10, wherein the step (c3) further comprises:
(c34) a step in which the artificial intelligence unit sets a plurality of second diagonal lines that connect from a vertex located on another side of a lower part among vertices of the third reference rectangle to an edge of the largest upper tibia;
(c35) a step in which the artificial intelligence unit sets a fourth contact point in which the shortest second diagonal line among the plurality of second diagonal lines contacts the largest upper tibia; and
(c36) a step in which the artificial intelligence unit sets the third baseline by connecting the third contact point and the fourth contact point.

12. The method for of claim 5, wherein the step (c4) comprises:
(c41) a step in which the artificial intelligence unit receives the plurality of malleolus CT images transmitted from the big-data unit;
(c42) a step in which the artificial intelligence unit finds a malleolus CT image in which the largest malleolus is captured among the plurality of malleolus CT images based on pre-learned malleolus CT images;
(c43) a step in which the artificial intelligence unit sets a fourth reference rectangle that contacts the largest malleolus in the malleolus CT image in which the largest malleolus is captured; and
(c44) a step in which the artificial intelligence unit sets a fourth baseline that bisects the fourth reference rectangle while being parallel to the long axis of the fourth reference rectangle.

13. An apparatus for measuring a femoral anterior angle and a tibial torsion angle by using artificial intelligence, the apparatus being configured for implementing the method according to claim 1.
